(19) United States
(12) Patent Application Publication
(10) Pub. No.: US 2017/0112888 A1
(43) Pub. Date: Apr. 27, 2017

(54) EYE LENS AND METHOD FOR MAKING SAME

(71) Applicant: HON HAI PRECISION INDUSTRY CO., LTD., New Taipei (TW)

(72) Inventor: HSIU-WEN CHIEN, New Taipei (TW)

(21) Appl. No.: 14/946,321

(22) Filed: Nov. 19, 2015

(30) Foreign Application Priority Data
Oct. 26, 2015 (TW) 104135156

(51) Int. Cl. A61K 36/64; B29D 11/00; A61K 9/00; A61F 2/16; G02B 1/04; A61K 31/353

(52) U.S. Cl. CPC A61K 36/64; G02B 1/043; A61K 31/353; A61K 9/0051; A61F 2/1624; B29D 11/00038; B29D 11/00134; B29D 11/00125; B29D 11/00009; B29D 11/00442; B29D 11/00432; A61F 2250/0067; B29K 2083/00

(57) ABSTRACT

An eye lens carrying eyebright extracts includes a matrix and a plurality of extracts dispersed in the matrix, the eyebright being released into the eye over a period of time to alleviate common disorders of the eye. Two methods for making the complete eye lens are also provided.

### FIELD

The subject matter generally relates to eye care.

### BACKGROUND

Eye lenses are widely used because they are very convenient. Wearing eye lenses for a long time may cause discomfort.

### BRIEF DESCRIPTION OF THE DRAWINGS

Implementations of the present technology will now be described, by way of example only, with reference to the attached figures.

FIG. 1 is a flow chart of making an eye lens.

FIG. 2 is another flow chart of making an eye lens.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented.

The term “comprising” when utilized, means “including, but not necessarily limited to”; it specifically indicates open-ended inclusion or membership in the so-described combination, group, series, and the like.

An embodiment of an eye lens may be a contact lens or an intraocular lens (IOL). The intraocular lens may be an accommodating intraocular lens (AIOL). The eye lens includes a matrix and a plurality of eyebright extracts dispersed in the matrix. The eyebright extracts are gradually released from the matrix. The eyebright extracts can treat or relieve inflammation of eyes (such as conjunctivitis, blepharitis, or styes), eye hemorrhage, or eye fatigue. Eyebright extracts also have functions of disinfecting, antioxidizing, improving vision, ocular function, relieving or reducing eye pouches, or relieving dark circles.

The eyebright extracts are extracted from the eyebright herb or euphrasia. It is well known that eyebright herb has medicinal value. The eyebright herb has many medicinal qualities. The eyebright can also treat eye congestion, keratitis, conjunctivitis, blepharitis, eye irritation, bronchitis, or sinus infection.

A method for preparing the eyebright extracts may include the following steps: (1) grinding the above-ground part of the eyebright herb or flower into powders; (2) adding the eyebright powders into a solvent to obtain a mixture, the solvent including water and ethanol; (3) filtering the mixture to remove solid matter in the mixture; (4) removing the ethanol from the mixture, thereby obtaining liquid eyebright extracts. The eyebright extract in liquid state may further be concentrated and dried to be a solid state. In this embodiment, the eyebright extract which is used is a liquid.

The eyebright can also be extracted by any other organic solvent or by water. The eyebright extract may also be purchased. For example, the company Xi'an DN Biology Co., Ltd offers eyebright extract for sale.

The eyebright extract mainly contains flavone (eyebright flavone).

The matrix has a mass percentage of about 90% to about 99.9999% of the total mass of the eye lens. The eyebright extracts have a mass percentage of about 0.0001% to about 10% of the total mass of the eye lens.

The eye lens may be a hydrogel lens or a silicone hydrogel lens. When the eye lens is hydrogel lens, the matrix is hydrogel matrix. When the eye lens is silicone hydrogel lens, the matrix is silicone hydrogel matrix. The hydrogel matrix may be prepared by irradiating a hydrogel precursor with ultraviolet light (UV) to copolymerize the hydrogel precursor. The hydrogel precursor includes a hydrated polymer, a photoinitiator and a cross-linking agent. The silicone hydrogel matrix may be prepared by irradiating a silicone hydrogel precursor with UV to copolymerize the silicone hydrogel precursor. The silicone hydrogel precursor includes a hydrated polymer, a photoinitiator, and a cross-linking agent.

When the matrix is a silicone hydrogel matrix, the hydrated polymer may be selected from a group consisting of methyl methacrylate (MMA) and hydroxyethyl methacrylate (HEMA). When the matrix is a silicone hydrogel matrix, the hydrated polymer may be selected from a group consisting of methyl methacrylate (MMA), hydroxyethyl methacrylate (HEMA), polydimethylsiloxane (PDMS), and tris(hydroxymethyl)aminomethane (TRIS).

The photoinitiator may be obtained from Chemical Industries Basel (CIBA) Corporation as a clear liquid under the trade name “Irgacure-1173”. The cross-linking agent may be ethyleneglycol dimethacrylate (EGDMA).

The hydrogel precursor and silicone hydrogel precursor may further include a hydrophilic monomer. The hydrophilic monomer is configured to improve the water-affinity and oxygen permeability of the hydrogel matrix and the silicone hydrogel matrix. When the matrix is a hydrogel matrix, the hydrophilic monomer may be selected from a group consisting of N-Vinyl-2-pyrrolidone (NVP), glycidyl methacrylate (GMA), and N,N-Dimethylacrylamide (DMMA). When the matrix is a silicone hydrogel matrix, the hydrophilic monomer may be N-Vinyl-2-pyrrolidone (NVP).

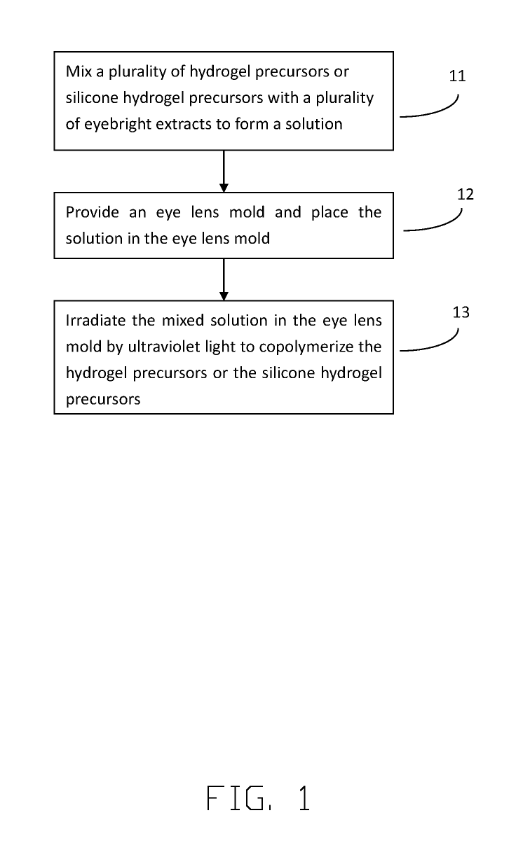

Referring to FIG. 1, a flowchart of a method for making the eye lens is presented. The example method is provided by way of example, as there are a variety of ways to carry out the method. The method can be carried out as illustrated in FIG. 1, for example. Each block shown in FIG. 1 represents one or more processes, methods, or subroutines carried out in the example method. Furthermore, the illustrated order of blocks is illustrative only and the order of the blocks can change. Additional blocks can be added or fewer blocks may be utilized without departing from this disclosure. The example method can begin at block 11.

At block 11, a plurality of hydrogel precursors or silicone hydrogel precursors are mixed with a plurality of eyebright extracts, thereby a solution is formed.

At block 12, an eye lens mold is provided, and the solution is placed in the eye lens mold.

At block 13, the solution in the eye lens mold is irradiated by UV, thereby the hydrogel precursor or the silicone hydrogel precursor are copolymerized to form a matrix with eyebright extracts dispersed in it.

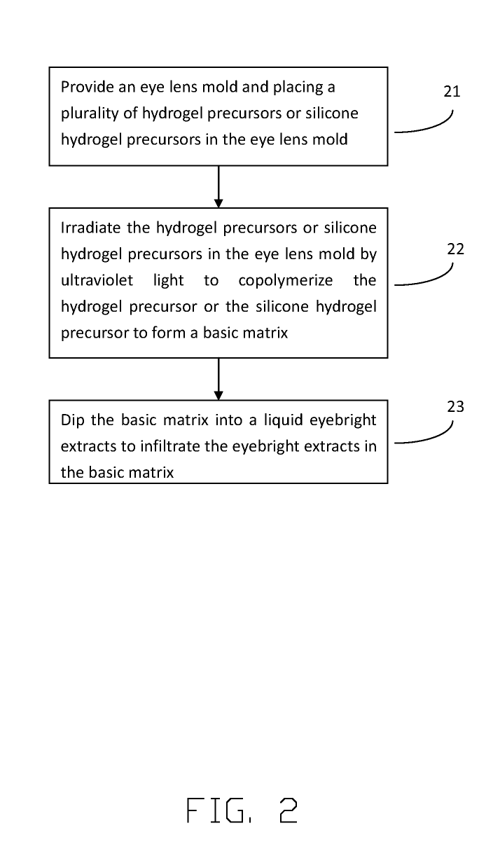

FIG. 2 shows a flowchart of another method for making the eye lens. The example method is provided by way of example, as there are a variety of ways to carry out the method. The method can be carried out as illustrated in FIG. 2. Each block shown in FIG. 2 represents one or more processes, methods, or subroutines carried out in the example method. Furthermore, the illustrated order of blocks is illustrative only and the order of the blocks can change. Additional blocks can be added or fewer blocks may be utilized without departing from this disclosure. The example method can begin at block 21.

At block 21, an eye lens mold is provided, and a plurality of hydrogel precursors or silicone hydrogel precursors are placed in the eye lens mold.

At block 22, the hydrogel precursor or the silicone hydrogel precursor in the eye lens mold are irradiated by UV, thereby the hydrogel precursor or silicone hydrogel precursor is copolymerized to form a basic matrix.

At block 23, the basic matrix is dipped into a liquid eyebright extracts for a period of time to infiltrate the eyebright extracts in the basic matrix.

### Example 1

The matrix of the eye lens was a hydrogel matrix. The matrix had a mass percentage of 98.5% of the total mass of the eye lens. The eyebright extracts had a mass percentage of 1.5% of the total mass of the eye lens.

The eye lens was made by the following steps: (1) HEMA, EGDMA, Irgacure-1173 and eyebright extract were mixed, thereby a solution was formed. The mass of the HEMA was 64 mg, the mass of the EGDMA was 3 mg, the mass of the Irgacure-1173 was 0.05 mg, and the mass of the eyebright extract was 1 mg. (2) An eye lens mold was provided, and the solution was placed in the eye lens mold. (3) The solution in the eye lens mold was irradiated by UV for 30 mins.

### Example 2

The matrix of the eye lens was a hydrogel matrix. The matrix had a mass percentage of 99.5% of the total mass of the eye lens. The eyebright extracts had a mass percentage of 0.5% of the total mass of the eye lens.

The eye lens was made by the following steps: (1) an eye lens mold was provided, and HEMA, EGDMA and Irgacure-1173 were mixed to form a hydrogel precursor. The hydrogel precursor was added into the eye lens mold, the mass of the HEMA was 64 mg, the mass of the EGDMA was 3 mg, the mass of the Irgacure-1173 was 0.05 mg, and the mass of the eyebright extract was 1 mg. (2) The hydrogel precursor was irradiated by UV for 30 mins, thereby a basic matrix was formed. (3) The basic matrix was dipped into liquid state eyebright extracts for a period of time and then taken out, the eyebright extract had a concentration within the lens of 10 mg/mL.

The embodiments shown and described above are only examples. Even though numerous characteristics and advantages of the present technology have been set forth in the foregoing description, together with details of the structures and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims.

What is claimed is:

1. An eye lens comprising:
a matrix; and
a plurality of eyebright extracts dispersed in the matrix.

2. The eye lens of claim 1, wherein the matrix has a mass percentage of about 90% to about 99.9999% of the total mass of the eye lens, the eyebright extracts have a mass percentage of about 0.0001% to about 10% of the total mass of the eye lens.

3. The eye lens of claim 1, wherein the eyebright extract comprises flavone.

4. The eye lens of claim 1, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor.

5. The eye lens of claim 4, wherein the hydrogel precursor or the silicone hydrogel precursor comprises a hydrated polymer, a photoinitiator, and a cross-linking agent.

6. The eye lens of claim 5, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

7. The eye lens of claim 5, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

8. The eye lens of claim 5, wherein the photoinitiator is Irgacure-1173, the cross-linking agent is ethyleneglycol dimethacrylate.

9. A method for making an eye lens comprising:
mixing a plurality of hydrogel precursors or silicone hydrogel precursors with a plurality of eyebright extracts to form a solution;
providing an eye lens mold and placing the solution in the eye lens mold; and
irradiating the mixed solution in the eye lens mold by ultraviolet light to copolymerize the hydrogel precursors or the silicone hydrogel precursors.

10. The method of claim 9, wherein the matrix has a mass percentage of about 90% to about 99.9999% of the total mass of the eye lens, the eyebright extracts have a mass percentage of about 0.0001% to about 10% of the total mass of the eye lens, the eyebright extract comprises flavone.

11. The method of claim 9, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor, the hydrogel precursor or the silicone hydrogel precursor comprises a hydrated polymer, a photoinitiator, and a cross-linking agent.

12. The method of claim 11, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

13. The method of claim 11, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

14. The method of claim 11, wherein the photoinitiator is Irgacure-1173, the cross-linking agent is ethyleneglycol dimethacrylate.

15. A method for making an eye lens comprising:
providing an eye lens mold and placing a plurality of hydrogel precursors or silicone hydrogel precursors in the eye lens mold;
irradiating the hydrogel precursors or silicone hydrogel precursors in the eye lens mold by ultraviolet light to copolymerize the hydrogel precursor or the silicone hydrogel precursor to form a basic matrix; and
dipping the basic matrix into a liquid eyebright extracts to infiltrate the eyebright extracts in the basic matrix.

16. The method of claim 15, wherein the matrix has a mass percentage of about 90% to about 99.9999% of the total mass of the eye lens, the eyebright extracts have a mass percentage of about 0.0001% to about 10% of the total mass of the eye lens, the eyebright extract comprises flavone.

17. The method of claim 15, wherein the matrix is a hydrogel matrix or a silicone hydrogel matrix, the hydrogel matrix is prepared by irradiating a hydrogel precursor with ultraviolet light to copolymerize the hydrogel precursor, the silicone hydrogel matrix is prepared by irradiating a silicone hydrogel precursor with ultraviolet light to copolymerize the silicone hydrogel precursor, the hydrogel precursor or the silicone hydrogel precursor comprises a hydrated polymer, a photoinitiator, and a cross-linking agent.

18. The method of claim 17, wherein the hydrogel precursor and silicone hydrogel precursor further comprises hydrophilic monomer, when the matrix is hydrogel matrix, the hydrophilic monomer is selected from a group consisting of N-Vinyl-2-pyrrolidone, glycidyl methacrylate, and N,N-Dimethylacrylamide; when the matrix is silicone hydrogel matrix, the hydrophilic monomer is N-Vinyl-2-pyrrolidone.

19. The method of claim 17, wherein when the matrix is a silicone hydrogel matrix, the hydrated polymer is selected from a group consisting of methyl methacrylate and hydroxyethyl methacrylate; when the matrix is a silicone hydrogel matrix, the hydrated polymer is selected from a group consisting of methyl methacrylate, hydroxyethyl methacrylate, polydimethylsiloxane, and tris(hydroxymethyl)aminomethane.

20. The method of claim 17, wherein the photoinitiator is Irgacure-1173, the cross-linking agent is ethyleneglycol dimethacrylate.
